(19) United States
(12) Patent Application Publication
(10) Pub. No.: US 2003/0216334 A1
(43) Pub. Date: Nov. 20, 2003

(54) Methods for inhibiting/treating hiv infections and aids related symptoms

(76) Inventors: Janos Aradi, Debrecen (HU); Thomas Bardos, Williamsville, NY (US); Joseph A. Dunn, Williamsville, NY (US)

Correspondence Address: Coleman Sudol Sapone, 714 Colorado Avenue, Bridgeport, CT, 06605-1601, US

(21) Appl. No.: 10/182,224

(22) PCT Filed: Jan. 5, 2001
(86) PCT No.: PCT/US01/00280

(51) Int. Cl. A61K 48/00; C07H 21/04

(52) U.S. Cl. 514/44; 536/23.1

(57) ABSTRACT

The present invention relates to a novel method for inhibiting the growth, elaboration and/or replication of HIV in human patients and to the prevention and treatment of human acquired immunodeficiency syndrome (AIDS) and other diseases caused by retroviral infection. In preferred aspects, the present invention provides a method for the use of a novel class of oligodeoxynucleotides consisting of 4-thio-2′-deoxyuridylate units for the prevention and treatment of both wild-type and drug-resistant Human Immunodeficiency Virus 1 (HIV), the causative pathogen of AIDS.

### FIELD OF THE INVENTION

The present invention relates to a novel method for inhibiting the growth, elaboration and/or replication of HIV in human patients and to the prevention and treatment of human acquired immunodeficiency syndrome (AIDS) and other diseases caused by retroviral infection. More particularly, in preferred aspects, the present invention provides a method for the use of a novel class of oligodeoxynucleotides consisting of 4-thio-2′-deoxyuridylate units for the prevention and treatment of both wild type and drug-resistant Human Immunodeficiency Virus 1 (HIV), the causative pathogen of AIDS.

### BACKGROUND OF THE INVENTION

Recent progress in the development of drugs against AIDS has led to the discovery of essentially two types of anti-HIV agents based on their mode of action; that is reverse transcriptase inhibitors and protease inhibitors. However, the utility of currently available anti-HIV drugs is limited because of their potential for toxicity as well as the emergence of resistant viral strains. In addition, none of these drugs cure this mostly fatal disease but only provide a palliative or delaying effect. Therefore, more effective and less toxic drugs to treat HIV are absolutely required. In this respect, most recent clinical trials of a third type of agents that inhibit the entry of HIV into the cells appear to show encouraging results (DeClerq, Drugs R&D 2, 321-331; 1999). Theoretically such compounds may be used by topical administration for prevention of HIV infection, as well as by systemic administration for the therapy of the already established disease (Pilcher et al, AIDS 22, 2171-2173; 1999).

One of the crucial steps in the life cycle of retroviruses is the conversion of the viral genomic RNA into complementary DNA catalyzed by reverse transcriptase. Thus reverse transcriptase became one of the main targets in the development of drugs against HIV, the etiological agent of the acquired immune deficiency syndrome. Two clinically useful classes of reverse transcriptase inhibitors have emerged from this approach, that is the Nucleoside Reverse Transcriptase Inhibitors (NRTIs) and the Non-Nucleoside Reverse Transcriptase Inhibitors (NNRTIs). The NRTIs (i.e., zidovudine [AZT], lamivudine, didanosine, zalcitabine, stavudine) do not carry functional 3′hydroxyl groups. After conversion to triphosphosphates by cellular nucleoside and nucleotide kinases they are utilized as substrates by the viral DNA polymerase. They terminate the growing polynucleotide chain because no 3′ hydroxyl group is available for the next incoming nucleoside. In contrast the inhibitory activity of the NNRTIs (i.e., nevirapine [NVP], delaviridine, loviride) could be attributed to specific interactions with the enzyme, i.e., by binding to the enzymes active site [Carpenter et al, JAMA, 276, 146-157 (1996); Balzarini et al, Proc. Natl. Acad. Sci., 93, 13152-13157 (1996); Gulick et al, N. Eng. J. Med., 337, 734-739 (1997)].

A new approach toward the development of anti-HIV drugs based on the inhibition of reverse transcriptase involves oligonucleotide and polynucleotides functioning as antitemplates [Bardos et al, Antimicrob. Agents Chemother. 36, 108-114 (1992)]. These inhibitors are believed to act by binding to the template site of the polymerase enzyme and thereby displacing the natural viral template [Bardos and Ho, In: New Approaches to the Design of Antineoplastic Agents (Bardos, T J and Kalman, T I eds.) Elsevier/North Holland, Amsterdam, New York, 315-332 (1982); Vlassov et al, V. Mil. Biol. 24, 915-918 (1988); Bardos et al, Ann. N.Y. Acad. Sci., 255, 522 (1975)]). They differ from sequence specific oligonucleotide inhibitors (i.e., antisense and antigene) by being the only oligo- or polynucleotides that are directly aimed at the inhibition of the reverse transcriptase enzyme. In contrast, the antisense oligonucleotides are designed to interact with complimentary strands of certain messenger RNAs and thereby inhibit the synthesis of the corresponding proteins. The antigene, or triple-helix forming, oligonucleotides exert their inhibitory activities by interaction with the double helical DNA and thereby inhibiting nucleic acid synthesis [Heider and Bardos, In; Cancer Chemotherapy Agents, American Chemical Society Wash. D.C., 529-565, (1995)]. Thus the antitemplates, which are non-sequence specific and may be entirely homopolymers (i.e., composed from mononucleotide units derived from the same base), represent a unique class of retrovirus inhibitors based on their chemical structure and mode of action.

Previous work has shown that chemical thiolation of uracil and/or cytosine containing oligo- and polynucleotides leads to modification of a portion of the cytosine and uracil bases to give their corresponding 5-mercapto base analogs. These “partially 5-thiolated” oligo- and polynucleotides showed inhibitory activities against various RNA and DNA polymerases including particularly reverse transcriptases. It was demonstrated that they were competitive with functional templates and thus acted by an antitemplate mechanism [Tokes and Aradi, Biochim Biophys Acta, 1261, 115-120 (1995)]. A series of partially 5-thiolated-2′-deoxy cytidylates and 2′-deoxyuridylates were shown to inhibit the replication of HIV in primary human blood cells [Bardos et al, Antimicrob. Agents Chemother. 36, 108-114 (1992)]. The inhibitory activity appeared to increase with chain-length as well as the percentage of thiolation of the oligomers. However no completely thiolated homopolymers were available for investigation.

A second class of antitemplates was recently prepared by conversion of the 4-amino group of the cytosine bases to the corresponding 4-thio groups in 2′-deoxyoligocytidylates [Tokes and Aradi, FEBS Lett. 396, 43-46 (1996)]. The resulting 4-thiodeoxy uridylic acids showed competitive inhibition of the reverse transcriptase enzyme of HIV similar to that obtained with the partially 5-thiolated oligocytidylates. However, in this case, it was possible to prepare the fully thiolated derivatives, that is pure 4-thiodeoxy uridylic acids, which showed significantly higher inhibition of the enzyme than the products obtained from partial conversion of the cytidine bases.

### BRIEF DESCRIPTION OF THE FIGURES

FIG. 1 is a schematic representation of 4-thiolated oligodeoxyuridylates where n=10 to 80. In one case of one specific example of the present invention, n=33, i.e., the compound is a 35meric oligonucleotide, which is termed “Suligovir”.

FIG. 2 shows the stability of 4-thio-modified oligodeoxyuridylates determined by denaturing polyacrylamide gel electrophoresis after incubation of each compound for 0, 12 and 24 hours in tissue culture media containing 10% fetal calf serum. Bands of oligonucleotides and their breakdown products were visualized using a silver stain kit.

FIGS. 3A and 3B shows the result of experiments to study the chain length dependency of the inhibition of HIV replication by 4-thiolated oligodeoxyuridylates using the 20mer, 30mer and 35mer (Suligovir). Their inhibitory potential were determined in MT4 cells measuring the reverse transcriptase activity in the supernatant of the infected cell cultures. The shorter oligonucleotides were less active against HIV. In FIG. 3A, the concentration of oligonucleotides were given on the basis of mass. When the concentrations were 88 nM (corresponding to 1 μg/ml of Suligovir) the difference in inhibitory activity was even more pronounced (FIG. 3B).

FIG. 4 shows the results of toxicity studies of Suligovir in mammalian cells determined by studying the effects of this drug on the colony formation of human granulocyte-macrophage progenitor cells, which are a major target of agents that can damage bone marrow. Cytotoxicity was measured using the an automated tetrazolium-based colorimetric assay as previously described [Pauwels et al, J. Virol. Methods, 20, 309-321 (1988); Ikeda et al, Antiviral Res, 29, 163-173 (1996)]. As shown in FIG. 4, Suligovir did not considerably affect colony formation of granulocyte-macrophage progenitor cells, even at doses as high as 180 μg/ml.

### DESCRIPTION OF THE INVENTION

The present invention relates to the use of 4-thio-2′-oligodeoxyuridylic acids, preferably ranging in length from 12 to 82 nucleotide units (referred to as 12 mer or 82 mer), preferably 35 nucleotide units (35 mer) for the inhibition of the growth, replication and/or elaboration of HIV in humans, and thereby for the suppression or cure of AIDS and AIDS related symptoms. It further relates to the use of these compounds in topical application for the prevention of AIDS, due to their ability to inhibit the entry of HIV into the cells. The research data on which this invention is based demonstrate the activity of these compounds as inhibitors of the replication of both drug-sensitive (wild-type) and drug-resistant HIV, in particular, strains of HIV which are resistant to NRTIs, for example, zidovudine, lamivudine, didanosine, zalcitabine and stavudine, among others and NNTRIs, for example, nevirapine, delaviridine and loridine, among others.

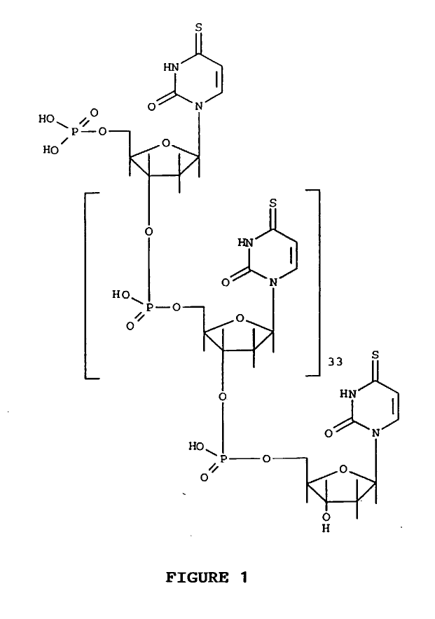

The 4-thio-2′-oligodeoxyuridylates are modified 2′-deoxyribonucleic acids in which the normal base components of DNA are replaced by 4-thiouracil. These compounds are schematically represented in FIG. 1, where n=10 to 80. In one specific example of this invention, n=33, i.e., the compound is a 35 meric oligo-nucleotide, which is named “Suligovir”. This is the preferred compound according to the present invention.

Compounds according to the present invention are 4-thio-2′-oligodeoxyuridylates as set forth in attached FIG. 1, where n is 10 to 80. Pharmaceutically acceptable salts of these oligodeoxyuridylates are clearly contemplated by the present invention. Pharmaceutical compositions according to the present invention comprise a therapeutically effective amount (anti-HIV effective amount) of one or more of the compounds according to the present invention, alone or preferably in combination with a pharmaceutically acceptable excipient, additive or carrier. Methods according to the present invention are directed to administering to an HIV, AIDS or other patient in need of such therapy a therapeutically effective amount of one or more pharmaceutical compositions according to the present invention to inhibit the growth or replication of HIV in the treated patient and ultimately to treat, eliminate or reduce the symptoms associated with AIDS. In addition, methods according to the present invention are directed to the topical or vaginal/cervical administration of pharmaceutical compositions containing 4-thio-oligodeoxyuridylates to healthy individuals in such form and amount as required to prevent their infection with HIV.

The following terms shall be used throughout the specification to describe the present invention.

The term “patient” is used throughout the specification to describe a human patient, to whom treatment, including prophylactic treatment, with the compositions according to the present invention is provided. For treatment of those infections, conditions or disease states which are specific for a specific human patient, the term patient refers to that specific patient.

The term “effective amount” is used throughout the specification to describe concentrations or amounts of compounds according to the present invention which may be used to produce a favorable change in the disease or condition treated, whether that change relates to the inhibition of the growth, replication and/or elaboration of the retrovirus (preferably, HIV), including, reducing the likelihood of or preventing a patient contracting an HIV infection or a reduction in severity or elimination of the symptoms associated with a condition or disease state, whether that condition or disease state is an HIV infection or AIDS

Certain of the compounds, in pharmaceutical dosage form, may be used as prophylactic agents for preventing a disease or condition from manifesting itself or substantially reducing the likelihood that a disease will manifest itself in a patient. In certain pharmaceutical dosage forms, the pro-drug form of the compounds according to the present invention may be preferred.

The present compounds or their derivatives, including prodrug forms of these agents, can be provided in the form of pharmaceutically acceptable salts. As used herein, the term pharmaceutically acceptable salts or complexes refers to appropriate salts or complexes of the active compounds according to the present invention which retain the desired biological activity of the parent compound and exhibit limited toxicological effects to normal cells. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, and polyglutamic acid, among others; (b) base addition salts formed with metal cations such as zinc, calcium, sodium, potassium, and the like, among numerous others including tertiary amines and related compounds. Base additional salts of the phosphate functionality of compounds according to the present invention are those which are clearly preferred for use in the present invention. Sodium and potassium salts of the present compounds are preferred.

Modifications of the active compound can affect the solubility, bioavailability and rate of metabolism of the active species, thus providing control over the delivery of the active species. Further, the modifications can affect the anti-HIV activity of the compound, in some cases increasing the activity over the parent compound. This can easily be assessed by preparing the prodrug form and testing its anti-HIV activity according to known methods well within the routineer's skill in the art.

The compounds of this invention may be incorporated into formulations for all routes of administration including for example, oral and parenteral including intravenous, intramuscular, intraperitoneal, intrabuccal, topical, transdermal, intracervical or intravaginal and in suppository form. Parenteral administration and in particuarly, IV administration is particularly preferred in the present method because HIV is a blood-borne disease and IV administration results in the compounds being adminstered at or near the site of infection.

Pharmaceutical compositions based upon these novel chemical compounds comprise the above-described compounds in a therapeutically effective amount for treating or preventing retroviral infections and other related diseases and conditions which have been described herein, optionally in combination with a pharmaceutically acceptable additive, carrier and/or excipient. One of ordinary skill in the art will recognize that a therapeutically effective amount of one of more compounds according to the present invention will vary with the infection or condition to be treated, its severity, the treatment regimen to be employed, the pharmacokinetics of the agent used, as well as the patient treated.

In the pharmaceutical aspect according to the present invention, the compound according to the present invention is formulated preferably in admixture with a pharmaceutically acceptable carrier. For therapeutic purposes, it is preferable to administer the pharmaceutical composition in parenteral, most preferably intravenous form, but a number of formulations may be administered via oral, parenteral, intramuscular, transdermal, buccal, subcutaneous, suppository or other route. Intravenous and intramuscular formulations are preferably administered in sterile saline. Of course, one of ordinary skill in the art may modify the formulations within the teachings of the specification to provide numerous formulations for a particular route of administration without rendering the compositions of the present invention unstable or compromising their therapeutic activity. In particular, the modification of the present compounds to render them more soluble in water or other vehicle, for example, may be easily accomplished by minor modifications (salt formulation, etc.) which are well within the ordinary skill in the art. It is also well within the routineer's skill to modify the route of administration and dosage-regimen of a particular compound in order to manage the pharmacokinetics of the present compounds for maximum beneficial effect to the patient.

In certain pharmaceceutical dosage forms, the pro-drug form of the compounds may be preferred. One of ordinary skill in the art will recognize how to readily modify the present compounds to pro-drug forms to facilitate delivery of active compounds to a targeted site within the host organism or patient. The routineer also will take advantage of favorable pharmacokinetic parameters of the pro-drug forms, where applicable, in delivering the present compounds to a targeted site within the host organism or patient to maximize the intended effect of the compound.

The amount of compound included within therapeutically active formulations according to the present invention is an effective amount for treating the infection or condition. In its most preferred embodiment, the present compounds, and in particular, the 35 meric oligo-nucleotide Suligovir, are used for treating HIV infections and AIDS related symptoms. In general, a therapeutically or prophylactically effective amount of the present preferred compound in dosage form usually ranges from slightly less than about 0.025 mg./kg. to about 10 g./kg. or more, preferably about 2.5-5 mg/kg to about 250 mg/kg of the patient or considerably more, depending upon the compound used, the condition or infection treated and the route of administration, although exceptions to this dosage range may be contemplated by the present invention.

Administration of the active compound may range from a preferred continuous (intravenous drip) to several oral administrations per day (for example, Q.I.D.) and may include oral, topical, parenteral, intramuscular, intravenous, sub-cutaneous, transdermal (which may include a penetration enhancement agent), buccal, vaginal, and suppository administration, among other routes of administration. Intravenous administration is clearly the preferred route of administration.

To prepare the pharmaceutical compositions according to the present invention, a therapeutically effective amount of one or more of the compounds according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. For parenteral, intravenous formulations, the carrier will usually comprise sterile water or aqueous sodium chloride solution, though other ingredients including those which aid dispersion may be included. Of course, where sterile water is to be used and maintained as sterile, the compositions and carriers must also be sterilized. Injectable suspensions may also be prepared, in which case appropriate liquid carriers, suspending agents and the like may be employed.

The present compounds may be used to treat human patients with an HIV infection, including AIDS patients. These patients can be treated by administering to the patient an effective amount of one or more of the compounds according to the present invention or its derivative or a pharmaceutically acceptable salt thereof optionally in a pharmaceutically acceptable carrier or diluent, either alone, or in combination with other known pharmaceutical agents, depending upon the disease to be treated). This treatment can also be administered in conjunction with other anti-HIV agents.

Alternatively, the present compound can be used on healthy persons who are potentially exposed to HIV infection. In such case, it is preferred to use the composition in a pharmaceutical formulation as a suppository or as an ointment.

The active compound is included in the pharmaceutically acceptable carrier or diluent in an amount sufficient to deliver to a patient a therapeutically effective amount for the desired indication, without causing serious toxic effects in the patient treated.

The compound is conveniently administered in any suitable unit dosage form, including but not limited to one containing 1 to 10,000 mg (10 g.), preferably 5 to 500 mg or more of active ingredient per unit dosage form.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

The following examples are provided to illustrate the present invention.

### EXAMPLE 1

Synthesis of 4-Thiolated Oligodeoxynucleotides. The thiolated oligonucleotides were prepared and characterized according to our earlier described method based on the original procedure of Ueda and co-workers (Miura et al, J. Biochem. 73, 1279-1284 (1973); Miura et al, Chem. Pharm Bull, 28, 3415-3418, (1980) and Miura et al, Biochim Biophys. Acta, 739, 181-189 (1983)]. Briefly, 1.5-3.5 mg of oligodeoxycytidylate (dCₙ, where n=20, 30 and 35) was dissolved in aqueous pyridine and treated with liquid H₂S for 9 days at 55° C. The concentration of modified oligonucleotides was determined measuring the phosphate content of the stock solution [Tokes and Aradi, FEBS Lett. 396, 4346 (1996)].

All of the unmodified oligodeoxycytidylate used as starting materials were synthesized in our laboratory using a Gene Assembler Plus (Pharmacia) automated DNA synthesizer on 1.3 μmol or 10.0 μmol supports.

It is obvious by those trained in the art that the 4-thiolated oligodeoxynucleotides themselves can be synthesized by alternative methods, in particular by automated direct synthesis [Oligonucleotide Synthesis; A Practical Approach, Gait, M. J. ed., IRL, Wash. D.C., (1984); Uhlmann et al, Chem Rev., 90, 544-584 (1990); Sonveaux, V., Biorg. Chem., 14, 274-325, (1986); Sonveaux, V., Methods Mol. Biol., 26, 1-71 (1994)] using a solid support and a commercially available 4-thio-2′-deoxyuridylic acid properly protected as the phophoamidite derivative, e.g. 5′-dimethoxytrityl-2′deoxy-4-4(2-cynaoethylthio)-uridine,3′-[2-cynaoethyl)-(N,N-diisopropyl)]-phosphoamidite.

### EXAMPLE 2

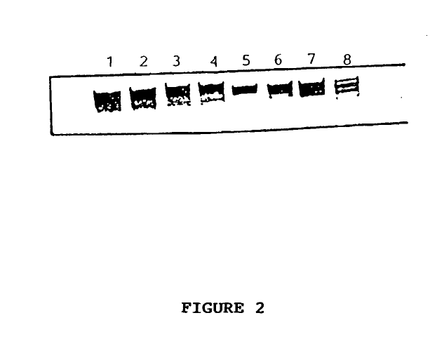

Stability of 4-Thiolated Oligodeoxynucleotides. The stability of 4-thio-modified oligodeoxyuridylates was determined by denaturing polyacrylamide gel electrophoresis after incubation of each compound for 0, 12 and 24 hours in tissue culture media containing 10% fetal calf serum Bands of oligonucleotides and their breakdown products were visualized using a silver stain kit (Pharmacia) according to the manufacturer's instruction. As shown in FIG. 2, Suligovir was surprisingly stable under these conditions, while its (dC)₃₅ analog was significantly degraded after 24 hours.

### EXAMPLE 3

Anti-HIV Activity of the 4-Thiolated Oligodeoxynucleotides. Two types of assay systems were used to study the anti-HIV activity of the 4-thiolated oligodeoxynucleotides. All experiments were performed in triplicate at least twice. In some studies, the inhibitory activity of the 4-thiolated oligomers against HIV was determined using a reverse transcriptase assay employing supernatants from the culture media of the Human T cell culture line, MT-4, infected with HIV-1(IIIB) (Hoffman et al, Virology, 147, 326-335, (1985)]). Briefly, 2.5×10⁵ MT-4 cells were infected with virus stock containing 100 TCID₅₀ (tissue culture infectious doses that establishes a productive infection in 50% of the parallel cultures) per ml. After 96 hours of growing the infected cells at 37° C. in a %5 humidified CO₂ atmosphere in RPMI 1640 media containing 10% fetal calf serum (complete media), the cells were removed by centrifugation and the reverse transcriptase activity was determined in 50 ml aliquots of the supernatants using poly (A).poly(dT)₁₆ as the template primer and [³H]dTTP as the monomer substrate. The radioactive product was collected by filtration and quantified by liquid scintillation counting. The oligonucleotide inhibitors were added to the cultures of MT-4 cells 1 hour post infection or as indicated. Appropriate control samples were prepared without inhibitors.

In other inhibition studies, a syncytium-forming microassay was used to quantify the amount of infectious HIV in supernatant fluid of virus infected Human CEM-SS cell cultures [Nara et al, AIDS Res Hum Retroviruses, 3, 283-302 (1987); Toth et al, J. Virol, 69, 2223-2232 (1995)]. Individual wells of a 96-well microtiter plate were coated with 50 μl of a 50 μg/ml ploy-L-lysine solution (PLL), allowed to stand at room temperature for 1 hour, after which residual PLL was removed by two washes with PBS. CEM-SS cells suspended in complete RPMI 1640 were plated into each well at a final concentration of 5×10⁴ cells/well. The cells were allowed to attach for 30 min at 37° C. Following this, supernatants were removed from the wells and replaced with 100 l fresh complete media. The wells were examined for the presence of adherent syncytium-forming units (SFU's) at 4 days post infection. Control CEM-SS cells were always included as a reference source when evaluating syncytium formation.

In preliminary experiments we treated MT-4 and CEM-SS cells with 20 μg/ml of Suligovir four hours before infection resulting in essentially complete inhibition of viral replication determined 96 hours after infection. The oligonucleotide did not affect the proliferation and morphology of either cell type cells. In order to optimize and study the effect of schedule of the treatments, Suligovir was added to the culture of MT-4 and CEM-SS cells in two concentrations (1 and 4 μg/ml) at various time points as compared to the infection. The results of these experiments are summarized in Tables 1 and 2. Even at 1 μg/ml (88 nM), Suligovir was very effective, inhibiting viral replication by more than 90% if it was added prior to infection or at the same time of infection. When 1 μg/ml was added 24 hours post-infection of MT-4 cells, only weak inhibition was observed (15%). The higher drug concentration (4 μg/ml) completely inhibited the replication of HIV showing a similar dependency on the schedule of treatment as in the case of the lower concentration. These results prove unequivocally that the 4-thiouridylate containing oligonucleotides have pronounced anti-retroviral effect.

### EXAMPLE 4

4-Thiolated Oligodeoxyuridylate Mode of Action.

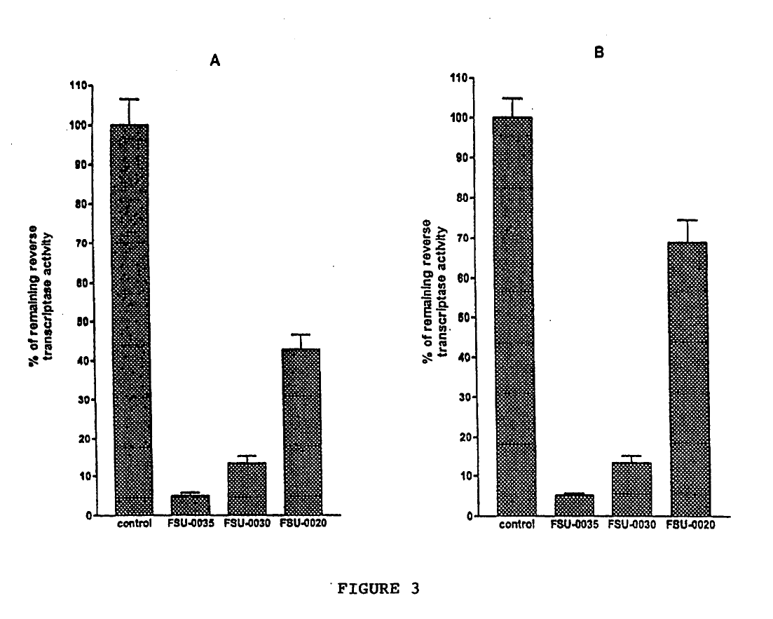

1) Inhibition of Reverse Transcriptase The 4-thiolated oligodeoxyuridylates are competitive inhibitors of the purified HIV-1 reverse transcriptase with respect to the template primer, fulfilling the requirement for an antitemplate inhibitor, i.e. the inhibitory oligonucleotide interacts with the free enzyme, competing for the template-binding site with its natural counterparts [Tokes and Aradi, FEBS Lett. 396, 43-46 (1996)]. In line with this mode of action, the inhibitory activity of the 4-thiolated oligodeoxyuridylates on purified reverse transcriptase depended on the chain length. Clearly more of the smaller molecules than of the larger molecules are required to fully occupy the templates site. To study the chain length dependency of the inhibition of HIV replication by 4-thiolated oligodeoxyuridylates, we synthesized the 20mer, 30mer and 35mer (Suligovir). Their inhibitory potential were determined in MT4 cells measuring the reverse transcriptase activity in the supernatant of the infected cell cultures (FIGS. 3A and B). The shorter oligonucleotides were less active against HIV. In FIG. 3A, the concentration of oligonucleotides were given on the basis of mass. In this case the total length of the added oligonucleotides in the assay system was essentially the same. When the concentrations were 88 nM (corresponding to 1 μg/ml of Suligovir) the difference in inhibitory activity was even more pronounced (FIG. 3B). When the chain length dependence of reverse transcriptase inhibition was studied on purified enzyme [Tokes and Aradi, FEBS Lett. 396, 43-46 (1996)], the inhibitory pattern was similar.

2) Inhibition of HIV-1 attachment and entry into target cells. In addition to the reverse transcriptase inhibition effects, further studies conducted at Serquest showed that Suligovir is a very potent inhibitor of HIV-1 attachment and entry into target cells (Table 3), 500 times more potent than the standard used in this assay, the polymer California Sky Blue (CSB). Suligovir inhibited virus entry (as measured by an attachment assay utilyzing the expression of the β-galactosidase enzyme measured by chemiluminescence in viral infected HeLa CD4 LTR β-gal cells as a measure of viral entry) with an IC₅₀ of 2 ng/ml, which corresponded to approximately 0.2 nM. When evaluated as an inhibitor of gp120 binding to CD4 in the same system (but measuring the amount of viral p24 antigen associated with the cells as a read-out), the IC₅₀ of Suligovir was 3 ng/ml (0.3 nM). In a different cell-to-cell fusion assay (in the absence of virus) employing HeLa CD4 LTR β-gal and HL2/3 cells, the IC₅₀ of Suligovir was 8.75 μg/ml (875 nM). The data strongly suggests that Suligovir is targeting an event early in virus-cell association, before or at gp120-CD4 interaction.

### EXAMPLE 5

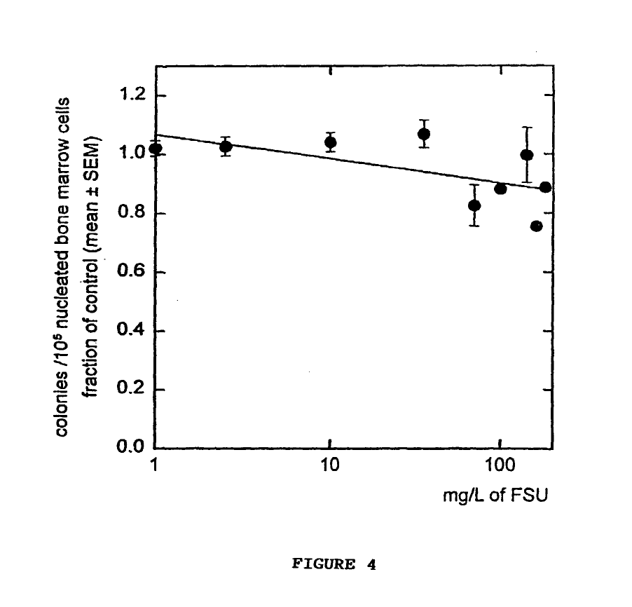

Cytotoxicity. The toxicity of Suligovir to mammalian cells was determined by studying the effects of this drug on the colony formation of human granulocyte-macrophage progenitor cells, which are a major target of agents that can damage bone marrow. Cytotoxicity was measured using the an automated tetrazolium-based colorimetric assay as previously described [Pauwels et al, J. Virol. Methods, 20, 309-321 (1988); Ikeda et al, Antiviral Res, 29, 163-173 (1996)]. As shown in FIG. 4, Suligovir did not considerably affect colony formation of granulocyte-macrophage progenitor cells, even at doses as high as 180 μg/ml.

### EXAMPLE 6

Effect on Drug-Resistant Mutants. Since the 4-thiolated oligodeoxynucleotides are inhibitors of reverse transcriptase, the range of effects of Suligovir on different mutants of HIV was determined compared to nucleoside-type reverse transcriptase inhibitors (NRTI) represented by AZT, and to non-nucleotide reverse transcriptase inhibitors, (NNRTI) represented by Nevirapine. Viral cytopathogencity was determined by Serquest Inc., a Division of Southern Research Institute, Gaithersburg Md. using the tetrazolium-based calorimetric assay, and the data were analyzed using a program developed by the Southern Research Institute. A number of viruses were used in these studies, which include some that are resistant to both the NNRTIs and NRTIs. None of these viruses was resistant to Suligovir as shown in the summary Tables 4 and 5.

D. Summary:

This series of thiolated oligonucleotides are chemically well-defined molecules that inhibit the replication of HIV by either or both of the following two mechanisms: 1) by inhibition of HIV reverse transcriptase at the template binding site; 2) inhibition of the entry of HIV into cells. They show a broad range of activity against various HIV mutants and therefore should be useful in the treatment of drug-resistant AIDS. In addition, they may be used in topical formulations as a prophylactic agent to prevent the spread of AIDS.

TABLE 1
Inhibition of HIV replication by (s⁴dU)₃₅;
Effects of various protocols of treatments in the presence of
1 and 4 μgf/ml inhibitor as determined by measuring reverse
transcriptase activity

| Time of | INHIBITION^{a} | |
|---|---|---|
| treatment | (RT activity: cpm^{b}; | |
| (hour) | % of inhibition) | |
| Concentration | 1 μg/ml | 4 μg/ml |
| −4 | 7377 ± 323 | 1722 ± 772 |
| | 93.6 ± 0.3 | 98.5 ± 0.7 |
| −4, +24 | 7279 ± 491 | 769 ± 373 |
| | 93.7 ± 0.4 | 99.3 ± 0.3 |
| −1 | 2506 ± 212 | 1149 ± 423 |
| | 97.8 ± 0.2 | 99.0 ± 0.4 |
| −1, +24 | 4062 ± 880 | 1213 ± 176 |
| | 96.5 ± 0.8 | 99.0 ± 0.2 |
| 0 | 2052 ± 274 | 1198 ± 345 |
| | 98.2 ± 0.2 | 99.0 ± 0.3 |
| 0, +24 | 3132 ± 177 | 1492 ± 477 |
| | 97.3 ± 0.2 | 98.7 ± 0.4 |
| +24 | 98547 ± 3114 | 87926 ± 2375 |
| | 15.2 ± 2.7 | 24.3 ± 2.0 |

^{a}All data are means ± standard deviation of the results of triplicate assays.
^{b}Control: 116 150 ± 4312 cpm

TABLE 2
Inhibition of HIV replication by (s⁴dU)₃₅;
Effects of various protocols of treatments in the presence of
1 and 4 μgf/ml inhibitor as determined by
syncytium-forming microassay

| Time of | Syncytium-forming units^{a,b} | |
|---|---|---|
| treatment (hour) | per 100 μl | |
| Concentration | 1 μg/ml | 4 μg/ml |
| −4 | 152 ± 29 | 41 ± 11 |
| −4, +24 | 138 ± 36 | 18 ± 7 |
| −1 | 52 ± 11 | 21 ± 4 |
| −1, +24 | 84 ± 27 | 26 ± 8 |
| 0 | 42 ± 7 | 22 ± 7 |
| 0, +24 | 67 ± 19 | 28 ± 4 |
| +24 | 1986 ± 227 | 1693 ± 177 |

^{a}All data are means ± standard deviation of the results of triplicate assays.
^{b}Control: 2423 ± 312 SFU

TABLE 3
Inhibition of Attachment, Fusion and gp120/CD4
Binding by Suligovir

| | | | Antiviral Activity (μg/ml) | | |
|---|---|---|---|---|---|
| Assay^{a} | Compound | Control | IC₅₀ | TC₅₀ | TI |
| Attachment | CSB^{b} | + | 1.19 | >10 | 8.4 |
| | Suligovir | | 0.002 | >100 | >50,000 |
| Fusion | CSB | + | 0.67 | >10 | >15 |
| | Suligovir | | 8.75 | >100 | >11.4 |
| gp120/CD4 | CSB | + | 1.2 | >10 | 8.3 |
| Binding | Suligovir | | 0.003 | >100 | >33,000 |

^{a}Each experiment was repeated at least once with similar results
^{b}CSB: Chicago Sky Blue. This is a control compound with a known effect on both virus attachment and fusion.

TABLE 4
Summary of Antiviral Results

| | AZT (μM) | | | Nevirapine (μM) | | | Suligovir (μg/ml) | | |
|---|---|---|---|---|---|---|---|---|---|
| Virus | IC₅₀ | TC₅₀ | TI | IC₅₀ | TC₅₀ | TI | IC₅₀ | TC₅₀ | TI |
| NL4-3 | 0.06 | 1 | 18 | 0.06 | 10 | 177 | 7.8 | 100 | 13 |
| K103N | 0.005 | 1 | 53 | 3.2 | 10 | 3.1 | 25.4 | 100 | 4 |
| Y181C | 0.01 | 1 | 100 | 1.03 | 10 | 9.78 | 6.7 | 100 | 15 |
| L1001 | 0.003 | 1 | 312 | 0.15 | 10 | 69 | 22 | 100 | 4.5 |
| 4XAZT | — | 1 | — | 0.05 | 1 | 21 | 35 | 100 | 4 |
| IIIB | 0.003 | 1 | 312 | 0.03 | 10 | 312 | 4.38 | 100 | 23 |
| DSP-R | 0.02 | 1 | 47 | — | 10 | — | 10.5 | 100 | 9.6 |

TABLE 5
Fold Resistance

| | AZT | | Nevirapine | | Suligovir | |
|---|---|---|---|---|---|---|
| | IC₅₀ | Fold | IC₅₀ | Fold | IC₅₀ | Fold |
| Virus | (μM) | Resistance | (μM) | Resistance | (μg/ml) | Resistance |
| NL4-3 | 0.06 | NA | 0.06 | NA | 7.78 | NA |
| (wild | | | | | | |
| type) | | | | | | |
| K103N | 0.02 | S | 3.2 | 53 (R) | 25.40 | 3.3 (S) |
| L100I | 0.0032 | S | 0.15 | 2.6 (S) | 22.2 | 2.8 (S) |
| 4XAZT | 1.0 | 16(R) | 0.05 | S | 24.5 | 3.1 (S) |
| YI81C | 0.01 | S | 1.03 | 17 (R) | 6.65 | S |
| HIV IIIB | 0.0032 | NA | 0.03 | NA | 4.38 | NA |
| (wild | | | | | | |
| type) | | | | | | |
| DPS-R | 0.02 | 6.7 (MR) | 10 | 333 (R) | 10.5 | 2.3 (5) |
| (Y181C) | | | | | | |

NA Not applicable
R: Resistant: Mutant/WT > 8
MR: Moderate resistance: mutant/WT 5 < n < 8
S: Sensative Mutant/WT < 5

1. A compound for use in treating retroviral infections comprising a compound according to the structure: 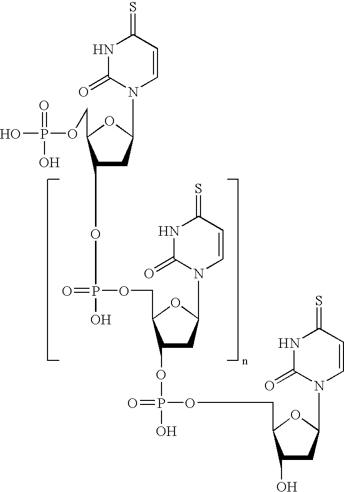
where n is from 10 to 80, or a pharmaceutically acceptable salt thereof.

2. The compound according to claim 1 where n is 33.

3. A pharmaceutical composition comprising an effective amount of a compound according to the claim 1, optionally in combination with a pharmaceutically acceptable additive, carrier or excipient.

4. The composition according to claim 3 where n is 33.

5. The composition according to claim 3 wherein n ranges from 20 to 50.

6. The pharmaceutical composition according to claim 3 or 4 in parenteral dosage form.

7. The composition according to claim 3-5 in topical dosage form.

8. The composition according to claim 3-5 adapted for vaginal or cervical delivery.

9. The composition according to claim 3-5 in suppository dosage form.

10. The composition according to claim 3-5 adapted for intravenous or intramuscular administration.

11. The composition according to claim 10 adapted for intravenous administration.

12. A method for treating HIV infections comprising administration to a patient in need of therapy a therapeutically effective amount of a composition according to any of claims 3-11.

13. A method for preventing or reducing the likelihood that a human exposed to HIV will contract an HIV infection, said method comprising prior to or during said

14. Use of a composition according to any of claims 3-11 to manufacture a medicament for the treatment or prevention of an HIV infection.
